(19) United States
(12) Patent Application Publication
(10) Pub. No.: US 2007/0079399 A1
(43) Pub. Date: Apr. 5, 2007

(54) Expression of desirable proteins in plant roots and cell cultures

(76) Inventor: Ilga Winicov, Waldoboro, ME (US)

Correspondence Address: NEEDLE & ROSENBERG, P.C., SUITE 1000, 999 PEACHTREE STREET, ATLANTA, GA, 30309-3915, US

(21) Appl. No.: 10/550,072

(22) PCT Filed: Mar. 22, 2004
(86) PCT No.: PCT/US04/08807

§ 371 Date: Nov. 17, 2006

Related U.S. Application Data
(60) Provisional application No. 60/456,714, filed on Mar. 21, 2003.

(51) Int. Cl. C12N 15/82; C12N 5/04; A01H 1/00

(52) U.S. Cl. 800/287; 800/288; 435/468; 435/419

(57) ABSTRACT

A new expression cassette for directing heterologous protein expression in plant roots has at least two portions, nucleotides encoding MsPRP2 promoter or a fragment thereof, said promoter or fragment comprising a portion of SEQ ID NO: 1; and nucleotides comprising a gene for a heterologous protein, operably linked to the MsPRP2 nucleotides. An expression cassette capable of directing heterologous protein expression in plant roots, comprising a) nucleotides encoding a promoter of MsPRP 2 or a fragment thereof; b) optionally nucleotides encoding a ribosomal binding site; c) optionally nucleotides encoding a secretion signal; and d) nucleotides encoding a heterologous protein, said protein nucleotides being operably linked to the MsPRP2 promoter nucleotides. The expression cassette further includes nucleotides encoding transcription factor Alfin1; the Alfin1 nucleotides are operably linked to another promoter such that the other promoter causes the transcription factor Alfin1 to be overexpressed. Also disclosed are plants and plant cells transfected with the preceding expression cassette. Also disclosed are methods for making plant cell-bound and secreted proteins. Seeds are made from transformed plant cells.

### FIELD OF THE INVENTION

This invention relates to a recombinant expression cassette and method for producing proteins in roots and plant cell cultures or secreted therefrom.

### BACKGROUND ART

Plant roots are adapted to accumulate water and nutrients from the soil and to provide these necessary ingredients for optimal growth and development of the entire plant. Plant roots also carry out specialized functions that contribute to overall plant yield and, in the case of root or tuber crops, constitute the essential plant yield. While proliferative growth of roots is determined by cell cycle regulation, and cyclin expression or plant hormones such as ethylene and auxin can enhance root growth, additional regulatory factors appear also to be necessary for new root growth.

Promoter strength and specificity are characteristics of endogenous gene expression and are essential for genetic engineering of heterologous gene expression. A limited number of promoters allow high levels of transgene expression, usually by ubiquitous and constitutive expression in all tissue types. The cauliflower mosaic virus (CaMV) 35S promoter is the most widely used promoter with these characteristics, but comparisons of different constitutive promoters for transgene expression are few. While diverse, tissue-specific genes have been characterized for roots, analysis and characterization of promoter elements have not revealed a uniform pattern among them for providing ubiquitous root expression. Cellular and developmental mechanisms of root growth and morphogenesis have been significantly elucidated in Arabidopsis. However, information about constitutive promoters for broad root specific expression has been less available.

The alfalfa MsPRP2 genomic sequence (Deutch and Winicov, 1995) and promoter region (Bastola et al., 1998) have been cloned. The MsPRP2 gene encodes a cell wall protein preceded by a signal sequence, and the transcript is expressed in a root-specific manner. The MsPRP2 transcript is constitutively expressed in roots and calli under normal growth conditions. In addition, the MsPRP2 promoter contains sequences that bind the alfalfa-root-specific transcription factor Alfin1 (Bastola et al., 1998). Overexpression of Alfin1 in transgenic alfalfa increased the expression of endogenous MsPRP2 gene from its own promoter in roots, but not in leaves and stems (Winicov and Bastola, 1999), which is consistent with the MsPRP2 promoter function in the root. Alfin1 and MsPRP2 overexpression enhanced overall root growth in transgenic alfalfa (Winicov, 2000).

Genetic engineering of plants to introduce agronomic or environmentally desired traits and produce desired biopharmaceuticals and nutraceuticals has been achieved with certain regulatory genes. Genetic engineering of plants is increasingly employed in agriculture to provide plant protection against a variety of stresses and pests, improve yield and add specific traits that would enhance the agricultural product. The potential for nutraceuticals in plants, such as enhanced levels of natural vitamins, their precursors or other beneficial organic compounds, seems limitless for human and animal consumption. A recent development in the area of medical molecular farming focuses on production of biopharmaceuticals, antibodies and edible vaccines in plants (Daniell et al., 2001). Both plant cells and tissue culture are being engineered to express molecules of commercial interest.

One of the most important factors for the successful introduction and expression of a heterologous gene is the promoter strength and specificity engineered in any transgene construct, since this is essential for high levels of expression of the transgene. Currently there are very few promoters available for development of transgenic plants. The highest levels of expression are usually achieved through ubiquitous expression in all tissues. Most transgene constructs for these purposes utilize promoters of virus origin to achieve the high levels of expression required for successful application of the transgenic plants for their specific purposes.

Gene transcripts have been cloned and are enhanced in salt-tolerant alfalfa cells and also are salt induced at the MRNA level in whole plants. One is Alfin1, which encodes a putative zinc-finger regulatory protein (Winicov, 1993). Another is MsPRP2, a single copy gene, which encodes a protein with a hydrophobic cysteine-rich carboxy terminus that could serve as a linker molecule between the cell wall and the membrane (Winicov and Deutch, 1994; Deutch and Winicov, 1995). Interestingly, both genes are expressed primarily in roots, and MsPRP2, too, is strongly salt inducible upon continued growth of the plants in 87 or 171 mm NaCl. Alfin1 is a transcription factor for the MsPRP2 promoter.

### SUMMARY

It has been discovered that the Alfin1 gene and the MsPRP2 promoter are a pair of regulatory genes from the alfalfa genome that can strongly promote the expression of autologous genes.

A new expression cassette for directing heterologous protein expression in plant roots has at least two portions: nucleotides encoding MsPRP2 promoter or a fragment thereof, comprising a portion of SEQ ID NO: 1; and nucleotides comprising a gene for a heterologous protein, operably linked to the MsPRP2 nucleotides. An expression cassette capable of directing heterologous protein expression in plant roots includes a) nucleotides encoding a promoter of MsPRP2 or a fragment thereof; b) optionally nucleotides encoding a ribosomal binding site; c) optionally nucleotides encoding a secretion signal; and d) nucleotides encoding a heterologous protein, said protein nucleotides being operably linked to the MsPRP2 promoter. The expression cassette further includes nucleotides encoding transcription factor Alfin1; the Alfin1 nucleotides are operably linked to another promoter such that the other promoter causes the transcription factor Alfin1 to be overexpressed. Also disclosed is a plant transfected with the preceding expression cassette. Also disclosed is a culture of plant cells with the preceding expression cassette. Also disclosed is a plant cell culture transfected with the preceding expression cassette.

In another embodiment, there is a method of producing a protein recombinantly in plant cells. The method has the steps of growing plant cells which have been transfected with an expression cassette having the following parts: nucleotides encoding a promoter of MsPRP2 or a fragment thereof, and nucleotides encoding the protein, said protein nucleotides being operably linked to the MsPRP2 promoter nucleotides and growing the transformed cells, during which the transformed cells produce the protein.

In yet another embodiment, there is disclosed a method of producing a secreted protein from plant cells. The method starts with growing plant cells which have been transfected with a protein-producing expression cassette which has the following parts: i. nucleotides encoding a promoter of MsPRP2 or a fragment thereof, ii. nucleotides encoding a secretion signal which are downstream from the MsPRP2 promoter or fragment thereof; and iii. nucleotides encoding the protein, said protein nucleotides being operably linked to the MsPRP2 promoter nucleotides. The next step is growing the transformed cells, during which the transformed cells produce the protein.

In yet another embodiment, there are disclosed seeds for plants producing a heterologous protein in its roots. The seeds are made from transgenic plant cells which have been transformed with nucleotides encoding a promoter of MsPRP2 or a fragment thereof and nucleotides encoding the protein, and optionally nucleotides encoding a plant secretion signal, the protein nucleotides being operably linked to the MsPRP2 promoter nucleotides and the secretion signal.

In yet another embodiment, there is disclosed a method of bioremediating a field, which calls for planting the seeds described above, with or without a secretion signal.

These and still further objects as shall hereinafter appear are readily fulfilled by the present invention in a remarkably unexpected manner as will be readily discerned from the following detailed description of an exemplary embodiment thereof, especially when read in conjunction with the accompanying drawings in which like parts bear like numerals throughout the several views.

### BRIEF DESCRIPTION OF THE DRAWINGS

FIG. 1 is a schematic of constructs for the expression of jellyfish green fluorescent protein (GFP) under the control of the cauliflower mosaic virus (CaMV) 35S promoter or the plant MsPRP2 promoter.

FIG. 2 shows the nucleotide sequences of the MsPRP2 genomic region upstream from the coding region, the last codon being the translation initiation codon.

FIG. 3 shows the nucleotide sequence of part of the constructs of FIG. 1. This includes the ribosomal binding portion and the signal sequence of MsPRP2 and the GFP coding sequence.

FIG. 4 is a three-dimensional bar graph showing the percents of transformants displaying different levels of GFP fluorescence, depending on the promoter and the presence of the MsPRP2 secretion signal sequence.

FIGS. 5A-5H are photographs that show GFP-fluorescent cells (FIGS. 5A-5D) and corresponding medium (FIGS. 5E-5H) to show GFP secretion from the cells. Indicated on the left are the constructs used to transform the cells.

FIGS. 6A-6D are photographs of GFP fluorescence in wild-type and Alfin1-overexpressing cells which have been transformed with the CaMV 35S or MsPRP2 promoter.

FIGS. 7A-7H are photographs of fluorescent roots. FIGs. 7A-7H show GFP fluorescence in root tip (FIG. 7A), mid-root including the epidermis and vasculature (FIGS. 7B and 7C) and root hairs and thick portion of root (FIG. 7D). FIGS. 7A-7D show wild type roots transformed with the MsPRP2promsig-GFP construct. FIGS. 7E-7H show the comparison of GFP fluorescence expressed from the CaMV 35Sprom-MsPRP2sig-GFP (FIGS. 7E and 7F) and the MsPRP2promsig-GFP (FIGS. 7G and 7H) constructs. FIGS. 7E-7H also show the effect of increased GFP fluorescence in LS-1 transformed plants that overexpress Alfin1 (FIGS. 7F and 7H) from the MsPRP2promsig-GFP construct (FIG. 7H).

FIGS. 8A-8F are photographs of FITC and the corresponding FITC/TRITC panels of transformed leaves illustrating the amount of GFP expressed from either the CaMV 35S promoter with no signal sequence (FIGS. 8A and 8E) or with signal sequence (FIGS. 8B and 8F); or the alfalfa MsPRP2 promoter-signal construct in the presence (FIGS. 8D and 8H) or absence (FIGS. 8C and 8F) of Alfin1 over-expression.

### DETAILS OF THE INVENTION

The present invention concerns the MsPRP2 promoter and its use in enabling high levels of expression of transgenes in plants, roots and cell cultures. Promoter sequences efficiently recognized by the cellular transcription and translation machinery for production of high levels of heterologous protein encoded by cDNA sequences cloned adjacent to this promoter are provided. These promoter sequences, originally cloned as a part of the genomic sequence of the MsPRP2 gene in alfalfa, in addition exhibit a two-fold increase in levels of transgene expression in plants overexpressing the gene Alfin1. Since the MsPRP2 promoter and Alfin1 are predominantly functional in roots in alfalfa, this invention will have important applications for genes targeted for expression in plant roots. The composite nucleotides sequences of the MsPRP2 promoter, MsPRP2 5′ untranslated sequence with its plant ribosome binding site for translation and the MsPRP2 signal sequence for cell wall targeting, provide an efficient means for transgene expression and secretion in cell cultures and plant roots.

The MsPRP2 promoter may be utilized to express heterologous genes in a variety of cell types from root tip to root hairs along the length of the root and all throughout the main body of the root as well as in plant cell cultures. This specificity to the roots contrasts with the ubiquitous expression pattern associated with the CaMV 35S promoter. The levels of heterologous gene expression in transgenic plant cells from the MsPRP2 promoter rival those from CaMV 35S promoter, particularly when the cells are overexpressing Alfin1.

In an important aspect of the invention, the MsPRP2 signal sequence is provided for promotion of secretion of GFP in transgenic plant roots, expressing the heterologous GFP as seen in the MsPRP2 promoter-signal-GFP constructs. The MsPRP2 signal sequence can be used in plant constructs alone or in combination with the MsPRP2 promoter. The MsPRP2 signal sequences cause secretion of heterologous proteins, to which they are linked, when expressed in plants from the MsPRP2 promoter or other promoters. There is less intracellular GFP in transformants that secrete GFP into the surrounding agar, due to the expressed MsPRP2 signal sequence as shown below. The high level of gene expression is illustrated infra by measurement of the final heterologous protein expression in transgenic plants.

These results support the novelty and utility of MsPRP2 promoter and signal sequences in heterologous gene expression and secretion in plant roots and cell cultures.

### EXAMPLES

The present invention may be better understood with reference to the accompanying examples that are intended for purposes of illustration only and should not be construed to limit the scope of the invention. All constructs were generated using standard molecular biology techniques.

To ensure measurement of equivalent expression of the GFP protein, which depends on transcription (promoter strength and efficiency), translation (efficiency of the 5′ untranslated region (UTR) that controls ribosome binding and translation initiation) and the transcript and protein stability after expression, comparable constructs were made for transgenic analysis. All constructs had the same coding sequence for GFP, the same 3′ UTR for mRNA stability and the same 5′ UTR from the MsPRP2 gene for translation efficiency. These constructs allowed for testing of the variables in promoter and signal efficiency; and in the case of the MsPRP2 promoter, the enhancement of expression due to Alfin1 overexpression as shown infra. All constructs were cloned in the binary vector pGA643 and introduced in alfalfa cells.

### EXAMPLE 1

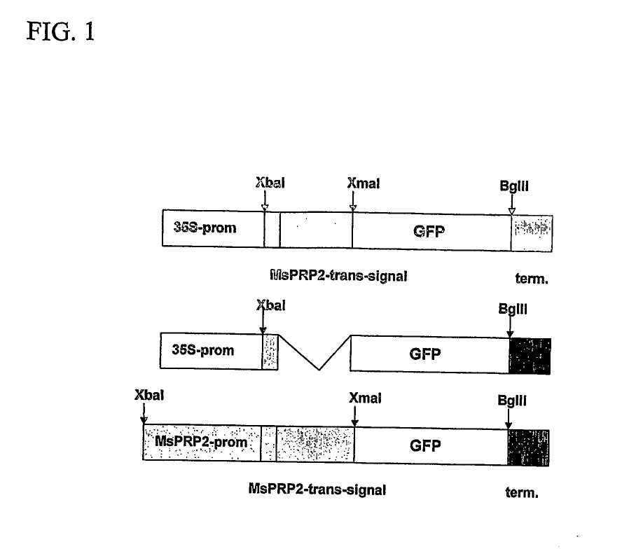

This example illustrates the preparation and use of constructs containing the MsPRP2 promoter and signal system for heterologous gene expression in plants. FIG. 1 shows the schematic and sequence of this ligation product, which contains a proline residue at the juncture from the primer-introduced XmaI site and allows the MsPRP2 promoter/signal fragment to be used as a cassette for subcloning with other heterologous genes for expression. The “35S-prom” block represents the CaMV 35S promoter; the adjacent dark block is the ribosome binding site of the MsPRP2 gene; the next dark block is the MsPRP2 signal sequence; the block labeled GFP encodes the green fluorescent protein; and the right-hand block is the transcription termination sequence. Where a line replaces the secretion signal sequence, there is no secretion signal sequence.

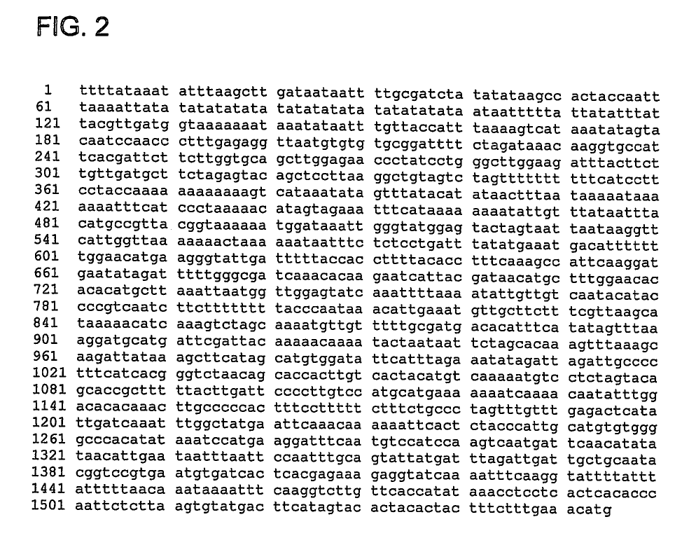

In summary, the GFP(S65T) sequence (Haseloff et al., 1997) was placed under transcriptional control of the −652 to +75 fragment of the MsPRP2 promoter (FIG. 2; SEQ ID NO: 1) or the CaMV 35S promoter. However, since the MsPRP2 (+1/+75 bp) sequence included a putative signal sequence for targeting FGP to the cell wall or secretion (Deutch and Winicov, 1995), we also cloned this putative signal sequence adjacent to GFP in one of the CaMV 35S promoter constructs (top construct of FIG. 1). This permitted us to monitor any effects of the signal sequence on the intracellular accumulation of the transgenically expressed GFP.

Constructs were made with a fragment of the MsPRP2 promoter and signal sequence (−652 to +75), where +1 is the A of ATG start of the MsPRP2 coding sequence (Bastola et al., 1998), by PCR using as Forward #1 primer (SEQ ID NO: 2) to introduce an XbaI site at the 5′ end and Reverse #1 primer (SEQ ID NO: 3) to introduce an XmaI site at the 3′ end of the molecule. All primers are listed in Table 1.

TABLE 1

| | | SEQ |
|---|---|---|
| | | ID |
| Primer | Sequence | NO: |
| Forward #1 | 5′GCTCTAGAGGATGCATGATTCGATTAG-3′ | 2 |
| Reverse #1 | 5′GGTCCCGGGCAAGCAAGAACAATGAG-3′ | 3 |
| Forward #2 | 5′GGACCCGGGGAGTAAAGGAGAAGAACTTTTCA | 4 |
| | C-3′ | |
| Reverse #2 | 5′GGAGATCTGAGCTCTTATTTGTATAGTTTC-3′ | 5 |
| Forward #3 | 5′GCTCTAGAACACTACACTACTTTCTTTGAACATG | 6 |
| | AGTAAAGGAGAAGAACTTTTCAC | |
| Forward #4 | 5′GCTCTAGAGTGTATGACTTCATAGTACAC-3′ | 7 |

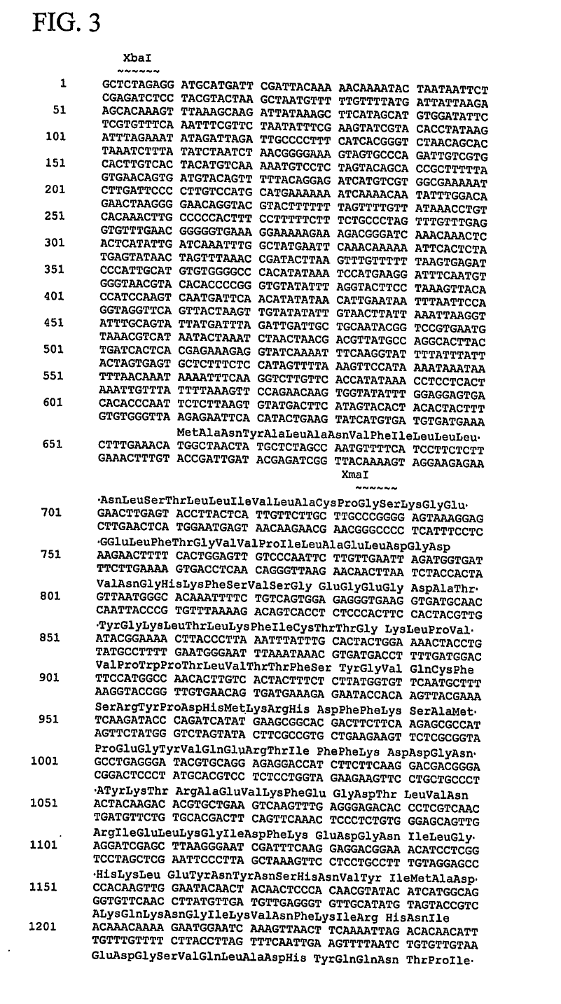

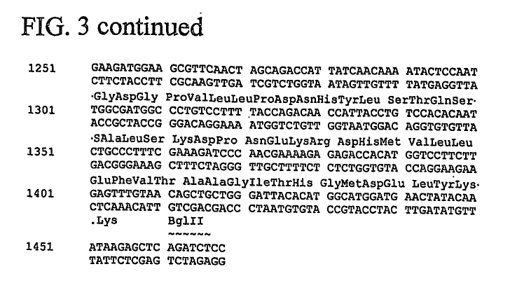

The heterologous GFP(S65T) gene was subcloned by PCR introduction of an XmaI site at the 5′ end with the Forward #2 primer (SEQ ID NO: 4) and introduction of a Bg1II site at the 3′ end with the Reverse #2 primer (SEQ ID NO: 5). The resultant PCR product of GFP(S65T) cDNA encoded the full GFP protein without the methionine at the NH₂ terminus and ended with lysine at the COOH terminus, without any KDEL sequences, which would retain this product in the cytoplasm/endoplasmic reticulum of the transgenic plants. The GFP and MsPRP2 promoter signal fragments were ligated at the XmaI site. FIG. 1 shows the schematic and FIG. 3 gives the sequence of this ligation product (GFP-MsPRPpromsig), which contains a proline residue at the juncture from the primer-introduced XmaI site and allows the MsPRP2 promoter/signal fragment to be used as a cassette for subcloning with other heterologous genes for expression. The GFP-MsPRPpromsig construct was further cloned in the binary vector pGA643 (An et al., 1988) and digested with XbaI and Bg1II (An et al., 1988).

The efficiency and strength of the MsPRP2 promoter was compared to that of the CaMV 35S promoter, which is one of the most commonly used and strongest promoters for expression of heterologous genes. The GFP(S65T) cDNA subclone was inserted into pGA643 digested with XbaI and Bg1II, adjacent to the 35S promoter of the vector. However, a fragment of the MsPRP2 5′ UTR (−28 to +1) was added to the 5′ end of the GFP(S65T) sequence by PCR using the Forward #3 primer (SEQ ID NO: 6) in order to provide an efficient plant ribosomal binding site for comparable translation of the two products from the 35S and MsPRP2 promoters. The Forward #3 primer (SEQ ID NO: 6) also introduced the XbaI site necessary for cloning in pGA643 and changes a T to an A in position 24 nucleotides upstream from ATG of the original MsPRP2 sequence. The reverse primer for this PCR reaction with GFP(S65T) was the same as described above for introducing the necessary Bg1II site for cloning in pGA643.

Additional control constructs were made to express GFP with the MsPRP2 signal sequence and the 5′ UTR under the control of the CaMV 35S promoter in pGA643. This was accomplished introducing an XbaI site at the 5′ end of the insert with the Forward #4 primer (SEQ ID NO: 7), which starts at position 618 in the GFP-MsPRPpromsig construct shown in FIG. 3. All constructs were verified by sequencing.

Agrobacterium transformation of alfalfa and selection of transformants was carried out as described previously (Winicov and Bastola, 1999). Transformation of alfalfa leaf discs from wild type parent (#1) or Alfin1-overexpressing LS-1 plants, callus growth and plant regeneration was carried out as described previously (Winicov and Bastola, 1999). Because LS-1 plants already carry the kanamycin resistance gene, transformant calli-expressing GFP were identified by fluorescence and were initially found to contain both transformed and untransformed cells. However, after three to four months of subculture, most of the GFP-construct-transformed LS-1 lines consisted of 80% or more GFP-expressing cells, possibly due to the double dose of kanamycin resistance gene after the second transformation.

### EXAMPLE 2

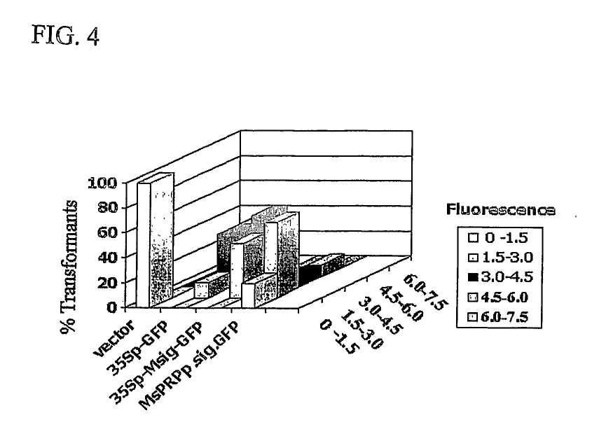

This example illustrates the expression and signal function of constructs, as prepared in Example 1 in alfalfa, and comparison of expression from constructs comprising the CaMV 35S promoter or MsPRP2 promoter. The results are illustrated in FIG. 4.

The endogenous MsPRP2 gene is expressed in alfalfa callus and roots, especially under stress or in Alfin1-overexpressing plants (Winicov and Bastola, 1999). However, use of the MsPRP2 promoter-signal sequence has not been previously established for expression of heterologous genes in alfalfa. We tested the strength of the MsPRP2 promoter by comparing the expression of GFP(S65T) from the MsPRP2 promoter with that of the CaMV 35S promoter in alfalfa control transgenic plants and in LS-1 plants. The CaMV 35S promoter is expressed ubiquitously throughout all tissues, roots and leaves alike.

From cultured, transformed LS-1 lines and #1 alfalfa lines, extracts were made by homogenizing 150 to 500 mg callus tissue in 1 ml of extraction buffer (10 mM Tris-EDTA, pH 8.0) for one minute at 4° C. using a plastic pestle driven by a homogenizer (approximately 800 rpm). Cell debris was removed by centrifugation (20 min at 16,000 g, 4° C.). Supernatant was used for fluorescence and protein measurements.

GFP expression in transformed alfalfa callus tissue extracts was quantitated by fluorescence measurements (λₑₓ=489 nm; λₑₘ=508 nm) using a SPEX FluoroMax™ Spectrofluorometer (Metuchen, N.J.). Fluorescence (FU) was calculated as photons X 10⁶/ml/g wet weight callus. Recombinant EGFP protein (Clontech, Palo Alto, Calif.) in 1 ml of extraction buffer was used to standardize dilutions in a linear range (0.05-0.4 μg/ml).

GFP fluorescence measurements in cellular extracts from these transformed calli showed significant variability among individually transformed lines. Low levels of residual fluorescence were seen in extracts from calli transformed with the empty vector, due to partial chloroplast development. Most of the transformed lines with both MsPRP2 and CaMV 35S promoters showed values well above those obtained with the vector control. Fluorescence data for transformants with each construct are shown in FIG. 4. The highest level of GFP fluorescence was obtained in transformants with the CaMV 35S promoter construct for the production of intracellular GFP. However, the presence of the MsPRP2 signal sequence adjacent to GFP decreased the level of intracellular GFP fluorescence in transformants with the CaMV 35S promoter.

These results suggested extracellular secretion of GFP with the MsPRP2 signal sequence, resulting in decreased intracellular levels of GFP. High levels of GFP fluorescence were also observed in calli transformed with the MsPRP2-promoter-signal construct. Significantly, nearly comparable levels of GFP fluorescence were obtained from cell extracts when GFP transcripts included the MsPRP2 signal sequence, with both the CaMV 35S and MsPRP2 promoters. These results indicated that the MsPRP2 promoter fragment used in these studies was indeed a strong promoter for heterologous gene expression. Since the MsPRP signal sequence could influence the intracellular accumulation of GFP, we further characterized its contribution to the GFP expression from both MsPRP2 and CaMV 35S promoters.

### EXAMPLE 3

A more detailed investigation of intracellular GFP expression and secretion due to the MsPRP2 signal sequence was carried out by confocal fluorescence microscopy of individual transformed cells and the agar medium surrounding transformed calli.

Fluorescent images of individual callus cells and roots and leaves of transgenic plants were taken with a Leica DM RBE epifluorescence microscope attached to a Leica TCS-NT confocal laser scanning head equipped with krypton-Argon laser (with 488 and 514 nm emission lines) and Leica 10× and 40× objectives. Callus cells were mounted on slides in Schenk and Hildebrand (1972) growth medium. Each specimen was scanned at 488 nm through a 40× (1.0 NA) oil-immersion objective lens. Serial scanned images in different planes were acquired using a fluorescence filter set (emission maximum 520 nm), and fluorescence intensity was quantified using the Leica TCS-NT software. Confocal Z series were collected through the entire cell volume to generate single-angle projections.

Images of root and leaf tissues were collected with a 512×512 pixel resolution with four passes through each of the eight equally spaced sections through the leaf. Two channels were used for detection of spectrally different signals of GFP and chlorophyll fluorescence. The aligned specimens were scanned with FITC-TRITC filter system with excitation maxima of 494 and 554 nm and emission maxima of 520 and 576 nm respectively. The FITC/TRITC filter combination was used for detection of green (GFP) and red (chlorophyll) fluorescent signals from transformed leaves. FITC alone was used for root tissue. Fluorescence intensity was digitally coded using 256 levels of gray.

FIGs. 5A-5D show the cellular fluorescence of alfalfa wild type (#1) cells transformed with the different constructs containing the CaMV 35S and MsPRP2 promoters with and without the MsPRP2 signal sequence. FIGS. 5E-5H show medium surrounding the transformed cells and GFP secretion in the presence of the MsPRP2 signal sequence. Qualitatively, the results from GFP fluorescence measurements by microscopy are similar to those obtained by fluorescence measurements on cellular extracts. Intracellular GFP levels are similar in cells from both CaMV 35S and MsPRP2 promoters when GFP transcript also encode the signal sequence.

### EXAMPLE 4

This example illustrates the effect of the signal sequence from the MsPRP2 promoter on secretion of the transgenic GFP prepared as in Example 1. GFP secretion in medium adjacent to calli was monitored with Nikon Eclipse TE 300 inverted microscope with ISEE software. Preparations were viewed through a 40× EL WD Plan Fluor/0.6 NA objective lens, using a fluorescence filter cube (FITC). Photographs were taken using Quantix® (Photometrics, USA) and/or Sony® CCD camera.

The surrounding-allus growth medium in FIGS. 5E and 5F showed no GFP secretion in the medium from vector or CaMV 35S-transformed cells, except as fluorescence from a cell remaining on agar. Other panels (FIGs. 5G and 5H) demonstrated that the combination of the MsPRP2 signal sequence with each promoter led to significant GFP secretion in the medium and confirmed that the MsPRP2 signal functions in secretion. This accounted for the lower levels of intracellular GFP from CaMV 35S promoter constructs whenever the MsPRP2 signal sequence was present. This experiment proved the functionality of the MsPRP2 signal sequence in heterologous protein secretion, thus enabling the use of the novel MsPRP2 sequences for expression of other heterologous genes in plants.

In addition, the MsPRP2 promoter confined most of the expression to roots in plants which express Alfin1 primarily in the roots. Alfin1-overexpressing plants would be highly useful in plant or cell systems designed to secrete the engineered products. The aspects of root directed and secreted products also differentiated the MsPRP2 sequences from other promoters used in plant transformation. Although a CaMV 35S promoter fragment (−90 to +1) has previously shown preferential regulation of genes in roots, this activity was very low compared to the full CaMV 35S promoter activity (Benfey et al., 1989) and therefore not very useful.

### EXAMPLE 5

This example was undertaken to determine if overexpression of Alfin1, encoding a putative transcription factor, could enhance the MsPRP2 promoter action on GFP Transgenic alfalfa LS-1 (previously transformed to overexpress Alfin1 (Winicov, 2000; Winicov and Bastola, 1999)) leaf discs were transformed with the MsPRP2-promoter-signal-GFP construct and the CaMV 35S-promoter-GFP construct as a control.

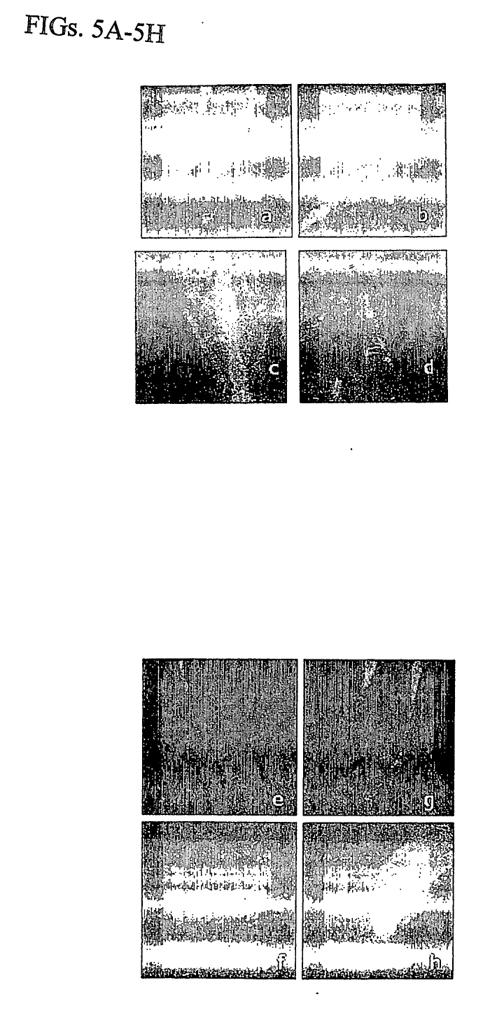

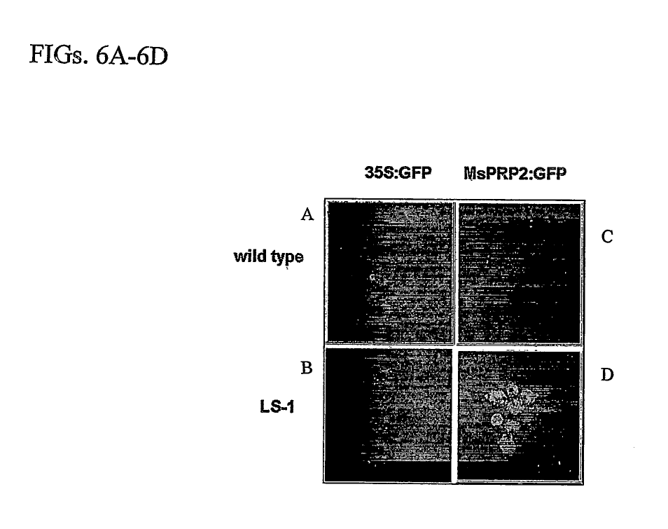

Intracellular GFP fluorescence was measured (as described above) in cells in both the wild type alfalfa (#1) and Alfin1-expressing LS-1 backgrounds. FIGS. 6A-6D show that concurrent overexpression of Alfin1 in the LS-1 plant promotes GFP expression from the MsPRP2 promoter, but not from the CaMV 35S promoter. Quantitative data for intracellular GFP fluorescence of many different individually transformed lines in each background and with each promoter construct are shown in Table 2. Differences in absolute values of intracellular GFP fluorescence between the two promoters are due to secretion, as mentioned above.

TABLE 2
Quantitative laser scanning confocal fluorescence (FU) measurements of cellular GFP
fluorescence in calli of independently transformed cell lines

| | | | % Transformed Lines within | | | | |
|---|---|---|---|---|---|---|---|
| | MsPRP2- | | Range of GFP Fluorescence* | | | | No. of |
| Promoter | Signal | Cells Type | 1-30 | 30-60 | 60-90 | >90 | Lines Tested |
| CaMV 35S | None | #1 (wt) | 8 | 8 | 42 | 42 | 12 |
| CaMV 35S | None | LS-1 | 18 | 59 | 12 | 11 | 17 |
| CaMV 35S | MsPRP2-sig | #1 (wt) | 67 | 33 | 0 | 0 | 6 |
| CaMV 35S | MsPRP2-sig | LS-1 | 80 | 20 | 0 | 0 | 5 |
| MsPRP2 | MsPRP2-sig | #1 (wt) | 100 | 0 | 0 | 0 | 20 |
| MsPRP2 | MsPRP2-sig | LS-1 | 66 | 28 | 6 | 0 | 18 |

*Mean GFP fluorescence as expressed per individual cell area

There were no significant differences in intracellular GFP fluorescence expressed in transgenic cells from the CaMV 35S promoter between the wild type (#1) and Alfin1-expressing LS-1 cells, indicating that the CaMV 35S promoter function was insensitive to changes in cellular levels of Alfin1 (Table 2). In contrast, GFP expression from the MsPRP2 promoter was higher in all tansgenic lines isolated from the LS-1 background overexpressing Alfin1 (FIG. 6D), when compared to the wild type alfalfa (#1) as shown in FIG. 6C and Table 2. The intracellular GFP increase could be seen despite the continuous secretion of GFP from the MsPRP2 construct. These results further confirmed the positive regulatory role for Alfin1 in gene expression from the MsPRP2 promoter and the fragment used in these studies.

These results support our evaluation of the MsPRP2 promoter as a strong promoter of heterologous genes, especially in presence of excess Alfin1.

### EXAMPLE 6

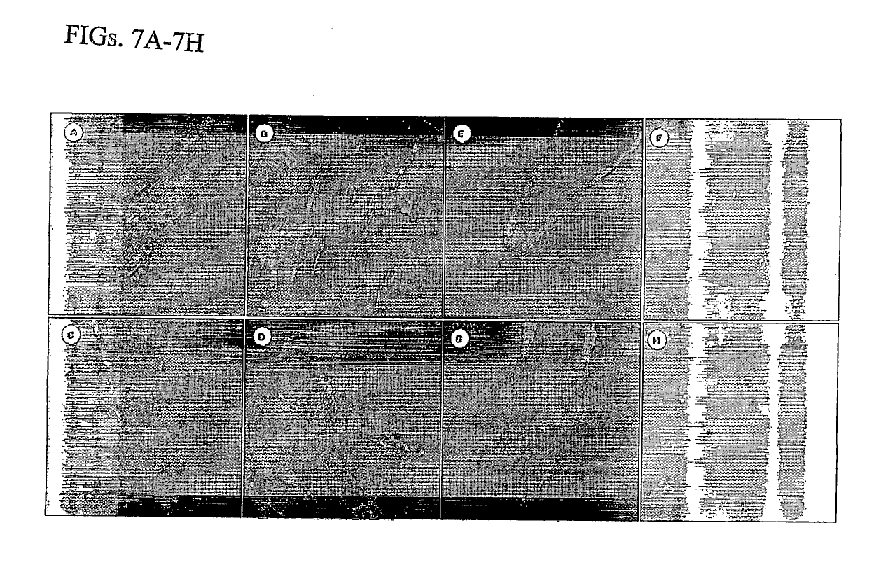

This example illustrates the tissue specificity of the MsPRP2 promoter for a variety of cell types along the length of the root. Plants were regenerated from the transformed calli in which GFP expression was measured as described above. The data are given in FIGS. 7A-7H.

FIGS. 7A-7D and 7G and 7H show strong, ubiquitous expression of GFP in the roots of alfalfa transformed with the MsPRP2-signal-GFP construct. GFP was detected in wild type plants (FIGS. 7A-7D and 7G) from root tip to the thick portions of the root and includes expression in root hairs (FIGS. 7A-7D). At the tissue level there was expression in the epidermis and central region of the root which includes the vascular system. Somewhat lower levels of GFP were seen in the cortex. The CaMV 35S control construct in these experiments also included the MsPRP2 sig-GFP sequence and is shown in FIGS. 7E and 7F, in order to eliminate differences in intracellular GFP due to secretion. FIG. 7E shows GFP expression in the wild type alfalfa and FIG. 7F show GFP expression in transformed LS-1 plant overexpressing Alfin1. GFP concentration at cell walls and the root surface indicated the continuous strong role of the MsPRP2-signal sequence in causing secretion of the GFP transgene product from the root. In addition, comparisons of FIGS. 7G-7H indicate that GFP expression from the MsPRP2 promoter was enhanced in the LS-1 plants overexpressing Alfin1, while no effect by Alfin1 is seen on the CaMV 35S expression of GFP (FIGS. 7E and 7F). These results were consistent with the results obtained with cell culture and shown in FIG. 4. These results show ubiquitous strong expression of GFP in diverse cell types from the MsPRP2 promoter used herein.

### EXAMPLE 7

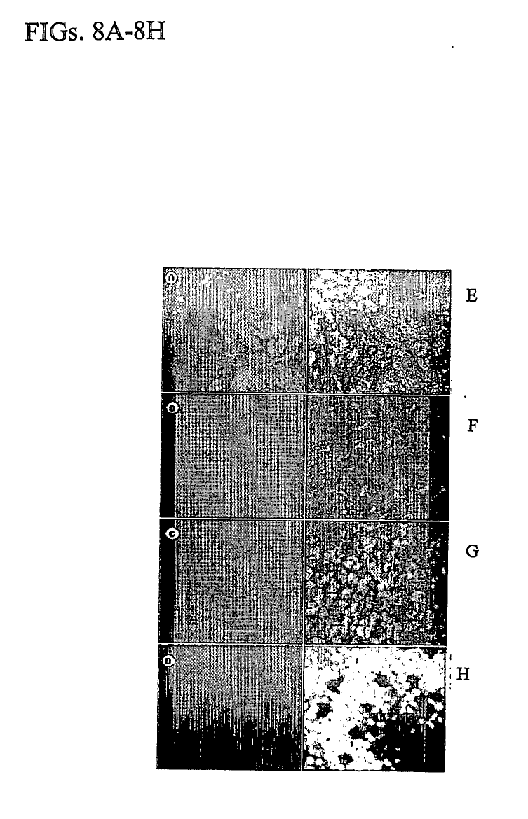

We analyzed closely the expression pattern from the MsPRP2-promoter-signal construct in leaves, since the endogenous MsPRP2 expression is root specific, and previous work with transgenic plants overexpressing Alfin1 in leaves (Winicov and Bastola, 1999) did not show endogenous MsPRP2 expression in leaves of those same transgenic plants. Thus, leaf expression would test both root specificity of our MsPRP2-promoter fragment and indicate whether Alfin1 was the only root-specific transcription factor required for GFP expression in the leaf from the constitutive MsPRP2 promoter. FIGS. 8A-8F show a comparison of GFP fluorescence in leaves of wild type (#1) plants expressing GFP from either the CaMV 35S promoter, CaMV 35S-promoter-MsPRP2-signal or the MsPRP2-promoter-signal construct. All transgenic plants tested for leaf expression of GFP showed strong expression of GFP in the roots.

Consistent with ubiquitous activity of the CaMV 35S promoter, we showed strong GFP expression in transgenic leaves of wild type plants with the CaMV 35S promoter, as shown in the FITC and the FITC/TRITC overlay panels of FIGS. 8A-8H. GFP accumulated in the cytosol and was excluded from chloroplasts, which contained highly fluorescent chlorophyll. However, accumulation of GFP in the presence of the MsPRP2-signal sequence appeared to be different (FIGS. 8A-8H). In the presence of the signal sequence, the level of GFP fluorescence was significantly decreased and localized, consistent with secretion from the cytoplasm. In the presence of the signal, we detected only localized GFP accumulation near vascular tissue in leaves, which may have been due to rapid transport or degradation within the leaf tissue. The appearance of localized expression of GFP in the presence of the MsPRP2 signal sequence was identical in transgenic plants from wild type (#1) and LS-1 origin plants (data not shown) and was also seen in some transformed calli.

In contrast, transgenic plants of wild type (#1) origin, with strong root expression of GFP from the MsPRP2 promoter, showed no detectable GFP fluorescence in their leaves (FIGS. 8C and 8G). In addition, transgenic LS-1 plants, which showed strong root expression of GFP from the MsPRP2 promoter, showed no detectable GFP fluorescence in their leaves, as can be seen in FIGS. 8D and 8H, even though these plants were shown to express Alfin1 in their leaves (Winicov and Bastola, 1999). These results were consistent with previous data showing no effect of Alfin1 overexpression on the endogenous MsPRP2 promoter in leaves of transgenic alfalfa (Winicov and Bastola, 1999).

### INDUSTRIAL APPLICABILITY

The expression cassette disclosed herein offers several commercially important advantages. It enables plant biologists to transform plants with a heterologous gene which is confined to the roots of a plant and which preferably functions in the root of the plant. With the secretion signal, the transformed plant secretes the heterologous protein from the roots.

The expression cassette is highly beneficial because it utilizes a plant-derived promoter(s), rather than a viral promoter, which has been alleged to contaminate adjacent crops.

Another benefit is the confinement of the protein expression to the roots. This can be used in multiple ways. First, the beneficial product can be directed to root crops, such as potatoes, yams and carrots. Second, above-ground agricultural products would be free of the root-bound proteins. Third, pests attacking the roots would be stopped by the heterologous protein.

A third benefit of this invention is rhizosecretion, a subset of molecular farming, which relies on the ability of plant roots or cultured cells or tissue (such as hairy roots) to exude valuable compounds. In contrast, most of the recombinant proteins and other valuable natural products used as fine chemicals, pharmaceuticals, crop protection compounds, cosmetic ingredients, etc., have been extracted from plants by using solvents and expensive purification methods. However, hydroponically cultivated plant roots and cultured plant cells and tissues can secrete the desirable products into simple media, from which purification is greatly simplified. Moreover, transgenic plants can produce and secrete proteins over a long time, as the plants need not be harvested to obtain the protein. Plants also are preferred sources of protein, as they are capable of carrying out acetylation, phosphorylation and glycosylation, as well as other post-translational protein modifications required for the biological activity of many eukaryotic proteins.

Although certain preferred embodiments and methods have been disclosed herein, it will be apparent from the foregoing disclosure to those skilled in the art that variations and modifications of such embodiments and methods may be made without departing from the spirit and scope of the invention.

All articles and books referenced herein are incorporated by reference.

### REFERENCES

1) An, G., Ebert, P., Mitra, A., Ita, S., and Vectors, B. (1988) Plant Molecular Biology Manual, Vol Section A (Dordrecht, The Netherlands, Kluwer Academic Publishers).
2) Bastola, D., Pethe, V., and Winicov, I. (1998) Alfin1, a novel zinc-finger protein in alfalfa roots that binds to promoter elements in the salt-inducible MsPRP2 gene, Plant Mol Biol 38, 1123-1135.
3) Benfey, P. N., Ren, L. and Chua, N-H. (1989) The CaMV 35S enhancer contains at least two domains which can confer different developmental and tissue-specific expression patterns. The EMBO Journal. 8, 2195-2202.
4) Daniell, H., Stephen, J., Streatfield, J., and Wycoff, K. (2001) Medical molecular farming: production of antibodies, biopharmaceuticals and edible vaccines in plants., Trends in Plant Sci 6, 219-226.
5) Deutsch, C. E. and Winicov, I. (1995) Post-translational regulation of a salt-inducible alfalfa gene encoding a putative Chimeric proline-rich cell wall protein., Plant Mol Biol 27(2), 411-18.
6) Haseloff, J., Siemergin, K. R., Prasher, D. C., Hodge S. (1997) Removal of a cryptic intron and subcellular localization of green fluorescent protein are required to mark transgenic Arabidopsis plants brightly, Proc Natl Acad Sci USA, 94(6), 2122-7.
7) Schenk and Hildebrand (1972) Medium and Techniques for induction and growth of monocotoledenous and dicot plant cell cultures, Can. J. Bot 50:199-204
8) Siemering, K., Golbik, R., Sever, R., and Haseloff, J. (1996) Mutations that suppress the thermosensitivity of green fluorescent protein, Current Biology 6, 1653-1663.
9) Winicov, I. (2000) Alfin1 transcription factor overexpression enhances plant root growth under normal and saline conditions and improves salt tolerance in alfalfa, Planta, 210(3), 416-22.
10) Winicov, I. (1993) cDNA Encoding Putative Zinc Finger Motifs from salt-tolerant alfalfa (Medicago saliva I.) cells, Plant Physiol, 102, 681-682.
11) Winicov, I., and Bastola, D. (1999) Transgenic overexpression of the transcription factor Alfin1 enhances expression of the endogenous MsPRP2 gene in alfalfa and improves salinity tolerance of the plants, Plant Physiol 120, 473-80.
12) Winicov, I. and Deutch, C. (1994) Characterization of a cDNA clone from salt-tolerant alfalfa cells that identifies salt inducible root specific transcripts, J Plant Physiol, 144, 222-228.

1. An expression cassette capable of directing heterologous protein expression in plant roots, comprising
a) nucleotides encoding MsPRP2 promoter or a fragment thereof, said promoter or fragment comprising a portion of SEQ ID NO: 1; and
b) nucleotides comprising a gene for a heterologous protein, operably linked to the MsPRP2 nucleotides.

2. An expression cassette capable of directing heterologous protein expression in plant roots, comprising
a) nucleotides encoding a promoter of MsPRP2 or a fragment thereof;
b) optionally nucleotides encoding a ribosomal binding site;
c) optionally nucleotides encoding a secretion signal; and
d) nucleotides encoding a heterologous protein, said protein nucleotides being operably linked to the MsPRP2 promoter nucleotides.

3. The expression cassette of claim 1 further comprising nucleotides encoding transcription factor Alfin1, the Alfin1 nucleotides being operably linked to another promoter such that the other promoter causes the transcription factor Alfin1 to be overexpressed.

4. A plant transfected with the expression cassette of claim 1.

5. A plant cell culture transfected with the expression cassette of claim 1.

6. A method of producing a protein recombinantly in plant cells, the method comprising:
a. growing plant cells which have been transfected with an expression cassette comprising:
i. nucleotides encoding a promoter of MsPRP2 or a fragment thereof; and
ii. nucleotides encoding the protein, said protein nucleotides being operably linked to the MsPRP2 promoter nucleotides; and
b. growing the transformed cells, during which the transformed cells produce the protein.

7. A method of producing a secreted protein from plant cells, the method comprising:
a. growing plant cells which have been transfected with an expression cassette comprising:
i. nucleotides encoding a promoter of MsPRP2 or a fragment thereof;
ii. nucleotides encoding a secretion signal which are downstream from the MsPRP2 promoter or fragment thereof; and
iii. nucleotides encoding the protein, said protein nucleotides being operably linked to the MsPRP2 promoter nucleotides; and
b. growing the transformed cells, during which the transformed cells produce the protein.

8. Seeds for plants producing a heterologous protein in its roots, the seeds comprising transgenic plant cells which have been transformed with nucleotides encoding a promoter of MsPRP2 or a fragment thereof, nucleotides encoding the protein, and optionally a plant secretion signal, the protein nucleotides being operably linked to the MsPRP2 promoter nucleotides and the secretion signal.

9. A method of bioremediating a field, the method comprising planting the transgenic seeds of claim 8 with or without the secretion signal.
